(19) United States
(12) Patent Application Publication
(10) Pub. No.: US 2012/0330095 A1
(43) Pub. Date: Dec. 27, 2012

(54) IMPLANTABLE INJECTION PORT

(75) Inventors: Dennis E. Van Epps, Goleta, CA (US); Alexei Goraltchouk, Rensselaer, NY (US)

(73) Assignee: ALLERGAN, INC., Irvine, CA (US)

(21) Appl. No.: 13/427,678

(22) Filed: Mar. 22, 2012

Related U.S. Application Data
(60) Provisional application No. 61/467,198, filed on Mar. 24, 2011.
(60) Provisional application No. 61/472,529, filed on Apr. 6, 2011.

(51) Int. Cl. A61F 2/04

(52) U.S. Cl. 600/37

(57) ABSTRACT

The present invention provides a access port assembly for use with a gastric band assembly. The system generally comprises an implantable access port and a porous material on the port for facilitating tissue ingrowth.

### CROSS REFERENCE

This application claims priority pursuant to 35 USC §119(e) to U.S. Provisional Patent Application No. 61/467,198, filed Mar. 24, 2011, and U.S. Provisional Patent Application No. 61/472,529, filed Apr. 6, 2011, both of which are hereby incorporated by reference in their entirety.

### FIELD

The present invention generally relates to medical systems and apparatus and uses thereof for treating obesity or obesity-related diseases, and more specifically, relates to access ports and methods for applying the same to bodily tissue.

### BACKGROUND

Adjustable gastric banding devices have provided an effective and substantially less invasive alternative to gastric bypass surgery and other conventional surgical weight loss procedures. Despite the positive outcomes of invasive weight loss procedures, such as gastric bypass surgery, it has been recognized that sustained weight loss can be achieved through a laparoscopically-placed gastric band, for example, the LAP-BAND® (Allergan, Inc., Irvine, Calif.) gastric band or the LAP-BAND APO (Allergan, Inc., Irvine, Calif.) gastric band. Generally, gastric bands are placed about the cardia, or upper portion, of a patient's stomach forming a stoma that restricts food's passage into a lower portion of the stomach. When the stoma is of an appropriate size that is restricted by a gastric band, food held in the upper portion of the stomach may provide a feeling of satiety or fullness that discourages overeating. Unlike gastric bypass procedures, gastric band apparatus are reversible and require no permanent modification to the gastrointestinal tract.

Medical implants, including gastric band systems, for performing therapeutic functions for a patient are well known. Such devices include pace makers, vascular access ports, injection ports (such as used with gastric banding systems) and gastric pacing devices. Such implants need to be attached, typically subcutaneously, in an appropriate place in order to function properly.

Many implantable medical devices are secured with sutures. For example, when inserting a gastric band and an associated access port, the associated access port may be sutured into place with sutures against the rectus muscle sheath. Such placement of the sutures is often challenging because the associated access port is placed below several inches of bodily tissue (e.g., fat), and suturing the associated access port often takes as long as placing the gastric band itself.

Additionally, the sutures can cause post surgical pain for the patient due to the inherent pulling and slight tearing of the tissue pieces by and adjacent to the suture.

Conlon, et al., U.S. Pat. No. 7,374,557, generally discloses self attaching injection ports comprising integral fasteners for subcutaneous attachment. However, Conlon does not disclose a porous coupling device.

U.S. Patent Publication Nos. 2005/0131352 and 2004/0254537 to Conlon, et al., also generally disclose a self attaching injection port comprising integral fasteners for subcutaneous attachment. Patent Publication Nos. 2005/0131352 and 2004/0254537 do not disclose a porous coupling device.

Olroyd, et al., U.S. patent application Ser. No. 12/772,039, filed on Apr. 30, 2010, and commonly owned herewith, discloses an implantable access port which may include a plurality of anchor assemblies and a mesh member attached to the base of the access port to facilitate implantation of the access port into a patient's body.

Honaryar, et al., U.S. patent application Ser. No. 12/904,422, filed on Oct. 14, 2010 and commonly owned herewith, discloses a system for attaching a fluid access port to a patient, where the system includes an implantable access port and a prefabricated mesh member attached to the access port to facilitate securing the access port to the tissue of the patient.

There remains a need for a procedure to implant medical devices in a quick, easy and efficient manner, which improve tissue integration.

### SUMMARY

The present invention, in one embodiment, provides an implantable access port device. The device may include an access port for use as a part of an adjustable gastric banding system, and a porous coupling member secured thereto. The coupling member may comprise a porous surface for facilitating tissue ingrowth and consequently, providing improved anchoring of the port to tissue. In one embodiment, the coupling member comprises a top surface securable to a base of the access port and a bottom surface structured to facilitate tissue ingrowth.

In one embodiment, the coupling member is an open-cell foam, for example, a open-cell silicone foam.

In some embodiments, the coupling member forms only portion of an exterior surface of the access port. In other embodiments, the coupling member forms the entirety of the exterior surface of the access port.

These and other aspects of the invention may be more clearly understood or appreciated by referring to the accompanying drawings and the detailed description.

### BRIEF DESCRIPTION OF THE DRAWINGS

FIG. 1 illustrates a partially exploded view of an access port device including an access port which may be a component of a gastric banding system, and a porous coupling member for facilitating tissue ingrowth and fixation of the access port in a gastric banding patient.

FIG. 2 illustrates a perspective view of the device shown in FIG. 1.

### DETAILED DESCRIPTION

The present invention relates to implantable medical devices and more specifically to access ports for gastric banding systems. Specifically, the present invention is directed to an implantable access port assembly for use as a part of a gastric banding system.

In various embodiments, the present invention utilizes porous coupling devices and/or combinations thereof to anchor or fix an access port to a patient. In some embodiments, a porous material (described in greater detail below) may be used to anchor an access port to a patient's tissue. For example, the porous material may encourage tissue ingrowth or tissue engagement through pores of the porous material. This use of porous materials to anchor the access port using the patient's own tissue engagement may obviate the need for sutures during implantation. For example, where access port implantation may occur using sutures, during recovery and beyond, movement of the patient may cause discomfort due to a pulling and slight tearing of the tissue around the implantation site. Use of a porous material, preferably an open-cell foam material, according to embodiments of the present invention, may eliminate or substantially reduce this discomfort.

Current endoscopic procedures typically require mechanical means for device fixation within the anatomy. In particular, within the realm of obesity intervention and therapies, many technologies exist which rely almost completely on fixating means such as staples, sutures, pivotal fasteners, mesh and the like to maintain fixation and positional stability of the device during the intended implantation duration.

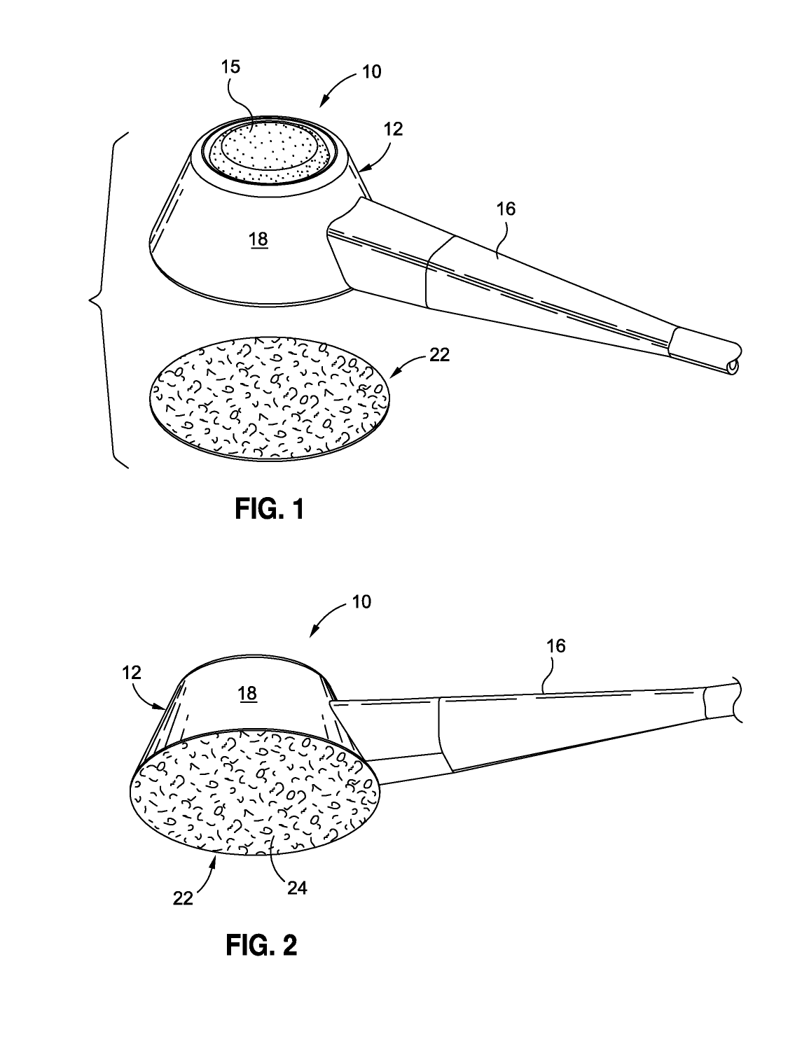

In accordance with the present invention, FIG. 1 and illustrate a simplified perspective view of an access port assembly 10 for use in conjunction with an implantable medical device, such as an implantable gastric banding system (not fully shown). Access port assembly 10 may comprise access port 12 including a fluid chamber (not visible in the Figures), a puncturable septum 15 for passing fluid into and out of the chamber via a needle, a tube 16 connecting the chamber to an inflatable portion of a gastric band, and a housing 18 generally circumscribing the septum 15 and chamber. The access port assembly 10 provides a convenient means for inflating and/or deflating a conventional, inflatable gastric band connected thereto via tube 16, thereby enabling adjustment of a size of a stoma or a level of restriction on a patient's stomach. The access port device 10 is generally fixed within the interior of a patient's body, preferably secured to a patient's abdominal muscle.

In accordance with one aspect of the present invention, the access port assembly 10 further a porous coupling member 22 secured to at least a portion of the housing 18 and providing a surface for facilitating tissue ingrowth and forming an anchoring surface 24 of the assembly 10. Although not shown in detail, when the device 10 is implanted, the anchoring surface 24 interfaces the abdominal muscle. In some embodiments, not shown, the anchoring surface 24 may further include additional mechanical anchoring mechanisms such as a latch, clip, aperture for sutures, hook, and/or the like, to further facilitate surgical coupling of the access port 12 to a patient. In other embodiments, the access port assembly 10 includes no such mechanical anchoring mechanisms, other than porous member 22.

Advantageously, the coupling member 22 facilitates tissue ingrowth and anchoring of the device 10 to a patient, with or without additional anchoring mechanisms. The coupling member 22 may comprise a porous material, for example, an open-cell foam, for example, a non-biodegradable, biocompatible foam, for example, a polymeric foam, for example, a silicone foam, or other suitable porous material capable of encouraging tissue ingrowth. The coupling member 22 may be of a suitable geometry to form the anchoring surface 24 and/or any other surface that interfaces the muscle fascia when the access port device is implanted in a patient.

The coupling member 22 is structured to facilitate or encourage healthy tissue ingrowth. In other words, the body's own tissue may grow into coupling member 22 and act as an anchoring and stability mechanism.

In a preferred embodiment, the coupling member 22 comprises a biodegradable or non-biodegradable foam. In some embodiments, the coupling member is a non-biodegradable foam, for example, a silicone foam. In some embodiments, the porous coupling member is an open cell foam.

For purposes of the following description, the term “pore size” can be defined as the average diameter equivalent of a void, and the term “interconnection diameter” can be define d as the average diameter equivalent of an intersectional plane between two voids.

In one embodiment, the porous coupling member is an open cell foam having a pore size of about 250 μm to about 1500 μm, and an interconnection diameter of about 100 μm to about 1200 μm.

In another embodiment, the porous coupling member is an open cell foam having a pore size of about 624 μm to about 408 μm and an interconnection diameter of about 133 μm to about 230 μm.

In yet another embodiment, the porous coupling member is an open cell foam having a pore size of about 408 μm to about 641 μm and an interconnection diameter of about 118 μm to about 325 μm.

In another embodiment, the porous coupling member is an open cell foam having a pore size of about 400 μm to about 550 μm and an interconnection diameter of about 150 μm to about 300 μm.

In yet another embodiment, the porous coupling member is an open cell foam having a pore size of about 470 μm and an interconnection diameter of about 210 μm.

In the embodiment shown, the porous coupling member 22 is disposed on only a portion of the outer surface of the housing 18. In other embodiments, not shown, the coupling member substantially entirely encapsulates the housing.

Advantageously, the porous coupling member 22, by promoting tissue ingrowth, may function to stabilize the access port 12 in the body and prevent movement or flipping thereof which can complicate gastric band adjustment.

Suitable porous coupling members, for example, suitable biocompatible open cell foams having a desired structure can be formed in any conventional manner suitable for making these materials.

Especially advantageous methods of making suitable coupling members in accordance with various embodiments of the invention are described in U.S. Patent Application No. 61/375,686, filed Aug. 20, 2010; U.S. Patent Application No. 61/387,074, filed Sep. 28, 2010; U.S. Patent Application No. 61/387,083, filed Sep. 28, 2010; U.S. Patent Application No. 61/387,115, filed Sep. 28, 2010; and U.S. patent application Ser. No. 13/015,309, filed Jan. 27, 2011; the entire disclosure of each of these documents being incorporated herein by this specific reference.

In one aspect of the invention, implantable components of gastric banding systems are provided wherein such components have an external surface at least a portion of which has an open cell texture, or which is covered by a porous material as described herein, and which can attain an optimal biological response. The open cell surface texture can be applied to the component by any suitable means known in the art.

In one embodiment the implantable components include the access port assembly 10 shown and described elsewhere herein, as well as the gastric band itself which contacts a stomach surface, and/or tubing that provides fluid connection between the access port and the gastric band. In accordance with this aspect of the invention, one or more of such components can include surfaces, such as porous open cell surfaces, which obtain an optimal biological response when the component is implanted in the body. In some embodiments, the implantable components include such porous, open cell surfaces that encourage tissue integration and less foreign body capsule formation and minimize the need for other anchoring features, relative to identical components having no open cell surfaces, for example, identical components having only relatively smooth surfaces. With respect to a gastric band component of a gastric banding system, the open cell foam surface on the band in accordance with the invention provides a means to anchor the band around the esophagus or adjacent the upper portion of the stomach while minimizing the foreign body reaction. The porous surface can be selectively positioned to localize desired sites of tissue integration.

The components in accordance with the invention may be made by providing an implantable member (e.g., an implantable access port, gastric band or other implantable medical device) and providing a porous material, for example, an open cell foam, as described herein. Next, a bonding substance is applied to a surface of the device and/or the porous material and the porous material is bonded to the device thereby forming an implantable assembly having at least one surface that encourages or promotes tissue ingrowth and/or other beneficial biological response.

The bonding substance may be, for example, a room temperature vulcanizing silicone (RTV) or high temperature vulcanizing (HTV) silicone. The bonding substance can be applied to the foam or felt described herein using any method known in the art, for example, brushing, spraying, dipping, curtain coating, vapor deposition methods can be used, casting methods can be used, injection molding and the like. The bonding substance can be cured using heat or any other means of aiding in curing known in the art.

After the porous material has been adhered to the surface of the implantable device, extra portions can be trimmed off to make a relatively smooth edge.

In some embodiments, the porous material is laminated to a surface of a implantable component of a gastric banding system. A dispersion of HTV silicone may be used as the adhesive between the implant and sheets of the porous material. In the process, the sheet is coated with a thin layer of HTV silicone and then placed in the bottom cavity. The smooth implant is then placed on top of the sheet in the cavity. The second sheet is coated with a thin layer of HTV silicone and applied on top of the smooth implant. The top piece of the cavity is then fixed in place pressing the two sheets together creating a uniform interface. The silicone adhesive is allowed to cure and then the excess material is cut off creating a uniform seam around the implant.

In another embodiment, a bonding surface is applied to the implant by dipping the implant into HTV silicone and then lamination of porous material onto the implant. The HTV silicone can be applied to the implant using any technique known to those skilled in the art, for example, by spraying curtain coating, and the like.

In some embodiments, the porous materials are applied only to portions of the implantable member. For example, only a portion of the entire surface of the implantable member is coated, for example, or only about 20%, about 30%, about 40%, about 50%, about 60%, about 70% about 80% or about 90% of the implantable member is covered with the porous material. In other embodiments, substantially all of the implantable member is covered with the porous material.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. An implantable access port assembly for use with a gastric banding system, the assembly comprising:
an access port including a fluid chamber, a septum, and a housing;
a flexible, porous coupling member secured to the housing and configured for enhancing tissue ingrowth and anchoring of the access port when the assembly is implanted in a patient.

2. The assembly of claim 1, wherein the porous coupling member is a silicone foam.

3. The assembly of claim 1, wherein the porous coupling member is an open cell foam.

4. The assembly of claim 1, wherein the porous coupling member is an open cell foam having a pore size of about 250 μm to about 1500 μm, and an interconnection diameter of about 100 μm to about 1200 μm.

5. The assembly of claim 1, wherein the porous coupling member is an open cell foam having a pore size of about 624 μm to about 408 μm and an interconnection diameter of about 133 μm to about 230 μm.

6. The assembly of claim 1, wherein the porous coupling member is an open cell foam having a pore size of about 408 μm to about 641 μm and an interconnection diameter of about 118 μm to about 325 μm.

7. The assembly of claim 1 wherein the porous coupling member is biodegradable.

8. The assembly of claim 1 wherein the porous coupling member is non-biodegradable.

9. The assembly of claim 1 wherein the porous coupling member is disposed on only a portion of the outer surface of the housing.

10. The assembly of claim 1 wherein the porous coupling member substantially entirely encapsulates the housing.

11. An implantable access port assembly for use with a gastric banding system, the assembly comprising:
an access port including a fluid chamber, a septum, and a housing;
a flexible, porous coupling member secured to a bottom surface of the housing and configured for enhancing tissue ingrowth and anchoring of the access port when the assembly is implanted in a patient, the porous coupling member being a open-cell silicone foam having a pore size of about 250 μm to about 1500 μm, and an interconnection diameter of about 100 μm to about 1200 μm.

12. The assembly of claim 11, wherein the porous coupling member is an open cell foam having a pore size of about 624 μm to about 408 μm and an interconnection diameter of about 133 μm to about 230 μm.

13. The assembly of claim 11, wherein the porous coupling member is an open cell foam having a pore size of about 408 μm to about 641 μm and an interconnection diameter of about 118 μm to about 325 μm.

14. The assembly of claim 11 wherein the porous coupling member is disposed on only a portion of the outer surface of the housing.

15. The assembly of claim 11 wherein the porous coupling member substantially entirely encapsulates the housing.
